(19) United States
(12) Patent Application Publication
(10) Pub. No.: US 2021/0046063 A1
(43) Pub. Date: Feb. 18, 2021

(54) NOVEL COMBINATION OF CRIZOTINIB WITH CHLOROQUINE FOR INHIBITION OF DIVERSE LUNG TUMORS

(71) Applicant: University of Kentucky Research Foundation, Lexington, KY (US)

(72) Inventors: Vivek M. Rangnekar, Nicholasville, KY (US); Ravshan Burikhanov, Lexington, KY (US)

(21) Appl. No.: 16/993,212

(22) Filed: Aug. 13, 2020

Related U.S. Application Data
(60) Provisional application No. 62/887,461, filed on Aug. 15, 2019.

(51) Int. Cl. A61K 31/4545; A61K 31/4706; A61P 35/00

(52) U.S. Cl. CPC A61K 31/4545; A61P 35/00; A61K 31/4706

(57) ABSTRACT

The presently-disclosed subject matter generally relates to methods for sensitizing chemotherapy resistant tumors using a synergistic drug combination. The presently-disclosed subject matter further relates to methods for sensitizing a drug resistant cancer cell to chemotherapy, comprising: contacting the drug resistant cancer cell with an effective amount of an agent capable of increasing cell surface expression of GRP78 and an agent capable of increasing soluble prostate apoptosis response 4 (Par-4).

### RELATED APPLICATIONS

This application claims priority from U.S. Provisional Patent Application No. 62/887,461 filed on Aug. 15, 2019 the entire disclosure of which is incorporated herein by this reference.

### GOVERNMENT INTEREST

This invention was made with government support under grant number R01 CA187273-01 awarded by the National Institutes of Health. The government has certain rights in the invention.

### TECHNICAL FIELD

The present invention relates to methods for sensitizing chemotherapy resistant tumors using a synergistic drug combination of agents capable of increasing cell surface expression of GRP78 and agents capable of increasing soluble prostate apoptosis response 4.

### BACKGROUND

Resistance to lung cancer treatment represents a significant, unsolved problem for which new solutions are urgently needed (1, 2). The tumor suppressor Prostate Apoptosis Response-4 (Par-4) is secreted by normal cells and induces apoptosis in lung cancer cells (3-6). Par-4 protein binds to the Par-4 receptor GRP78 at the surface of tumor cells and inhibits the growth of lung tumors in mouse models (4,5). The efficacy of tumor growth inhibition by Par-4 is dependent on the expression of GRP78 on the tumor cell surface (4, 5). On the other hand, most normal cells do not express GRP78 on their cell surface and are resistant to the action of secreted Par-4 (3-6). The instant invention: (a) elevates Par-4 secretion from normal cells, and (b) increases the expression of GRP78 on the tumor cell surface, in order to overcome resistance to lung cancer treatment. Chloroquine (CQ) used in cell culture and mice, and CQ derivative hydroxychloroquine (HCQ) used in a Phase 1 clinical trial, increased Par-4 secretion from normal cells, resulting in systemically elevated extracellular Par-4 levels and induced apoptosis in tumors (6, 7). CQ is a lysosomotropic drug that is known to inhibit the fusion of mature autophagosomes with lysosomes in the degradative, autophagic pathway (8). Findings indicated that CQ-inducible secretion of Par-4 from normal cells occurs independently of the autophagic pathway (6). CQ induces apoptosis in lung cancer cells via Par-4 secreted from normal cells and does not directly induce apoptosis on its own in the cancer cells (6, Preliminary Studies). CQ-inducible Par-4 secretion is blocked by brefeldin A (BFA), which inhibits post-Golgi trafficking of proteins via the conventional pathway (6). Remarkably, CQ induces Par-4 secretion from normal cells, but not from most of the lung cancer cells (6). Although CQ has been suggested to exert its anti-tumor effects by several mechanisms (8-11), Studies in mouse tumor models, using Par-4-null mice or a Par-4-neutralizing antibody, indicated that CQ induces apoptosis and inhibits lung tumors by a Par-4-dependent mechanism (6). CQ+CZT combination bypasses therapy resistance mechanisms to induce apoptosis and growth inhibition of lung cancer cell lines (FIG. 1). The instant invention demonstrates the combined effect of CQ and CZT (Crizotinib) on growth inhibition of lung cancer cell lines. The combined effect of Par-4 secretion induced by CQ in normal cells, and GRP78 upregulation induced by CZT in lung cancer cell lines that recapitulate the molecular traits of lung cancer.

Lung cancer is the leading cause of cancer-related deaths in the United States (14). Chemotherapy is the mainstay of advanced lung cancer treatment, but some lung tumors may be intrinsically resistant to chemotherapy, and even those that initially respond to treatment rapidly develop acquired resistance (1, 2). The tumor suppressor Par-4, which can inhibit the growth of both chemotherapy-sensitive and chemotherapy-resistant cancer cells (3-6). Par-4 protein functions both extracellularly and intracellularly to induce apoptosis in cancer cells (3-6, 15, 16). Extracellular (i.e., secreted) Par-4 binds to its receptor GRP78 found mainly on the surface of cancer cells, and selectively induces apoptosis of the cancer cells by caspase-8/caspase-3 activation (5, 6). The effect of extracellular Par-4 is dependent on intracellular Par-4, implying that the role of secreted Par-4 integrates with that of intracellular Par-4 in a cell surface GRP78-dependent manner (5). When activated, intracellular Par-4 functions in the cytoplasm by inhibition of the cell survival kinases or in the nucleus by transcriptional repression of cell survival genes (3, 4, 17). On the other hand, most normal cells lack GRP78 on their cell surface and are resistant to apoptosis by extracellular Par-4 (3-6). Recent studies have indicated that endothelial cells that are a part of the tumor microenvironment may also express cell surface GRP78 (18). Although oncogenic Ras downregulates Par-4 in selective cell lines, and Par-4-null mice show low frequency of lung tumors (23-25), a large majority of the lung tumors express Par-4 (26).

CQ induces Par-4 secretion from normal cells, elevates extracellular Par-4 levels systemically to primarily inhibit lung tumors by a Par-4-dependent mechanism, and not by autophagy-inhibition (6). Moreover, recombinant Par-4 protein, as well as the amino-terminal PAF domain of Par-4 inhibits lung cancer metastasis in mouse models (16, 27). Consistently, in Phase 1 clinical trials, HCQ elevated Par-4 levels in the plasma of cancer patients and induced apoptosis in their tumors that correlated with Par-4-induction and not with expression of p62/Sequestosome-1, a marker for autophagy inhibition (7). Although the concept of repurposing CQ or HCQ is not by itself novel, this study utilizes the novel concept of inducing Par-4 secretion from normal cells for inhibition of tumor growth and/or metastasis by CQ. Prior to studies with CQ, this drug was not known to induce the secretion of pro-apoptotic tumor suppressor proteins, such as Par-4. Normal cells, which have wild type p53, secrete Par-4 and that Par-4 induces paracrine apoptosis in cancer cells, regardless of their p53-status, while cancer cells express cell surface GRP78 (5, 6). By contrast, lung cancer cells did not show secretion of Par-4 in response to CQ (6). Interestingly, cancer cells that do not respond to Par-4 alone are readily sensitized to its apoptotic action by compounds such as PS1145 that inhibit NF-κB activity and induce the translocation of GRP78 to the cancer cell surface (28). Thus, cancer cells, including those that are Par-4-resistant, can be sensitized to the paracrine action of CQ-inducible Par-4 secretion from normal cells.

Although CQ may have multiple effects in vivo, including autophagy-inhibition, lysosomal catastrophe, normalization of blood vessels and repolarization of macrophages (8-11), studies using Par-4-null mice, recombinant Par-4 protein, as well as neutralization of CQ-induced Par-4 in mice with the Par-4 antibody strongly indicate that CQ-inducible secretion of Par-4 from normal cells is functionally essential for inhibition of lung tumor nodules (6). The multiple documented anti-tumor effects of CQ are therefore not expected to be a confounding factor in this study. CQ induces the conventional ER-Golgi secretory pathway that is BFA-sensitive and is dependent upon activation of p53 by CQ. Activated p53 then induces Rab8b, resulting in transport of the Par-4 protein cargo in Rab8b vesicles to the plasma membrane for secretion (6). Key proteins in this pathway are potential targets of p53 (6).

HCQ and CQ were studied in clinical trials that used these compounds either pre-operatively or in conjunction with chemotherapeutic agents or radiation, and the initial results of such CQ trials were encouraging despite the fact that the patients were not selected for inclusion in these trials based on any biochemical markers for sensitivity to CQ (29, 30). On the other hand, a recent study in a mouse pancreatic cancer model suggested that the gene-expression profile of pancreatic tumors not only rendered those tumors refractory to the benefits of CQ treatment but also promoted their growth, implying that CQ can induce prodeath or pro-survival functions dependent on the genetic context (31, 32). The precise genetic context that may render lung tumors sensitive to the effect of Par-4 secreted by CQ is not known. Cell surface GRP78 on lung cancer cells renders them sensitive to the action of secreted Par-4 (6, 28, and Preliminary Studies).

To maximize the effect of secreted Par-4, an FDA-approved drug library was screened for compounds that can complement the action of recombinant Par-4. One such compound that sensitizes lung tumor cells to the action of Par-4 is Crizotinib (CZT). The instant invention demonstrates that CZT, an inhibitor of ALK/MET/ROS1 kinases (12, 13), elevates Par-4 receptor GRP78 levels on lung cancer cells. Targeting the ALK oncogenic fusion protein has achieved remarkable clinical efficacy and enhanced the quality of life of patients. Although many patients respond well to initial treatment, CZT escape mechanisms lead to progressive disease. Acquired resistance to CZT may result from secondary mutations within the ALK kinase domain, amplification of the ELM4-ALK fusion gene, activation of SRC, epidermal growth factor receptor (EGFR)-mediated activation of HER family signaling, or downregulation of ALK, which can induce autophagy (33-35). Moreover, autophagy regulation has been suggested as a mechanism of growth inhibition by CZT in ALK-positive, as well as ALK-negative cells (36-38). The instant invention indicates that Par-4 used in conjunction with CZT induces apoptosis in lung cancer cell lines regardless of their ALK/MET/ROS1 or p53 status. Importantly, the effect of CZT in resistant lung cancer cells is restored by secreted Par-4 but not by direct treatment of the cancer cell cultures with CQ and CZT, implying that secreted Par-4 rather than autophagy-inhibition by CQ works in conjunction with CZT to induce apoptosis. Although, on the one hand, SRC activation confers resistance to CZT (33-35), on the other hand, SRC activation has been suggested to play a key role in inhibition of retrograde ER-Golgi pathway thereby promoting GRP78 translocation to the cancer cell surface (39). Interestingly, GRP78 is found mainly on the surface of cancer cells but not on most normal cells (5, 39-41). Cancer cells may utilize cell surface GRP78 for binding to diverse ligands for growth promotion (40, 41). The crux of the instant invention is to hijack the CZT resistance pathway by utilizing GRP78 on lung cancer cell surface to activate apoptosis by the ‘secreted Par-4-cell surface GRP78 axis’. Studies indicate that CZT induces SRC activation.

CQ is a potent inducer of Par-4 secretion from normal cells under conditions that do not show any normal cell death (6). As normal cells in any patient far outnumber cancer cells, Par-4 secretagogues such as CQ play a valuable role in elevating systemic and local levels of Par-4 protein and thereby induce paracrine apoptosis in primary or metastatic tumors (6, 7). Because Par-4 is a generic tumor suppressor that induces apoptosis in diverse tumor cells (3-7), the instant invention will have broad clinical and translational significance.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

One embodiment of the present invention is a method of sensitizing a drug resistant cancer cell to chemotherapy, comprising: contacting the drug resistant cancer cell with an effective amount of an agent capable of increasing cell surface expression of GRP78. In a further embodiment of the instant invention, the agent capable of increasing cell surface expression of GRP78 is Crizotinib. In some embodiments of the present invention, the method further comprises contacting the drug resistant cancer cell with an effective amount of an agent capable of increasing soluble prostate apoptosis response 4 (Par-4) in the drug resistant cancer cells.

In some embodiments of the present invention, the agent capable of increasing soluble Par-4 is selected from chloroquine, hydroxychloroquine, or combinations thereof. In another embodiment of the present invention, the drug resistant cancer cell is a lung cancer cell. In further embodiments of the instant invention, the drug resistant cancer cell is in a subject.

In some embodiments of the present invention, the agent capable of increasing cell surface expression of GRP78 is Crizotinib and the agent capable of increasing soluble Par-4 is selected from chloroquine, hydroxychloroquine, or combinations thereof. In further embodiments, the agent capable of increasing soluble Par-4 is chloroquine. In other embodiments, the drug resistant cancer cell is in a subject. In certain embodiments, the effective amount of Crizotinib is about 10 mg/kg, and the effective amount of chloroquine is about 25 mg/kg.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1A shows that CZT sensitizes lung cancer cells to apoptosis by Par-4. Lung cancer cells treated with vehicle, recombinant Par-4 (100 nM), CZT (1 μM), and a combination of CZT (1 μM) and Par-4 (100 nM) for 24 h, were subjected to: ICC analysis for active caspase-3, and apoptotic cells were scored by confocal microscopy. *P<0.01 by Anova or Student's t test.

FIG. 1B shows that CZT upregulates GRP78 expression on the surface of lung cancer cells. Lung cancer cells treated with vehicle or CZT (1 μM), for 24 h. Analysis for GRP78 at the cell surface. Mean+SD shown. *P<0.01 by Anova or Student's t test. P>0.05 by Anova.

FIG. 1C shows that CZT upregulates GRP78 expression on the surface of lung cancer cells but does not sensitize them to apoptosis by CQ in cell culture as CQ does not induce Par-4 secretion from the lung cancer cells. Lung cancer cells treated with vehicle, CQ (100 nM), CZT (25 μM), and a combination of CZT (1 μM) and CQ (25 nM) for 24 h. P>0.05 by Anova.

FIG. 2 shows both CZT alone and CQ+CZT causes significant inhibition of lung tumor growth. Mice were injected i.v. with LLC1 cells, and 24 h later injected i.p. with CQ (25 mg/kg body weight), CZT (10 mg/kg body weight), CQ+CZT (25 and 10 mg/kg, resp.) or vehicle once every day for 5 consecutive days. After 28 days, the lungs were scored for tumor nodules. *P<0.01 by Anova test.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a biomarker” includes a plurality of such biomarkers, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, width, length, height, concentration or percentage is meant to encompass variations of in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “subject” refers to a target of administration. The subject of the herein disclosed methods can be a mammal. Thus, the subject of the herein disclosed methods can include a mouse or human. The term does not denote a particular age or sex.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “upregulation” or “increasing expression” of a molecule such as GRP78 refers to an increase of the molecule can be by means of genetic manipulation or pharmacological intervention. Upregulation or increasing expression can be accomplished by direct upregulation of the molecule or upregulation of a positive regulator of the molecule, or down regulation of an inhibitor of the molecule.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level if or any particular patient will depend upon a variety of factors including the disorder being treated and the severity of the disorder; the specific composition employed; the age, bodyweight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, the term “drug resistant cancer cell” refers to cancer cells or cancer tissues, including whole cancerous tumors, that fail to respond to a chemotherapeutic agent. A response to a chemotherapeutic agent usually includes, but is not limited to, inhibition of cancer cell growth, proliferation, metastasis, or invasion.

As used herein, the term “chemotherapeutic agent” refers to pharmaceuticals used to treat cancer. As is well known in the art, such agents include but are not limited to: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine, and Vindesine.

### EXAMPLES

### CZT Elevates Cell Surface GRP78 and Sensitizes Lung Cancer Cells to Apoptosis by Extracellular Par-4

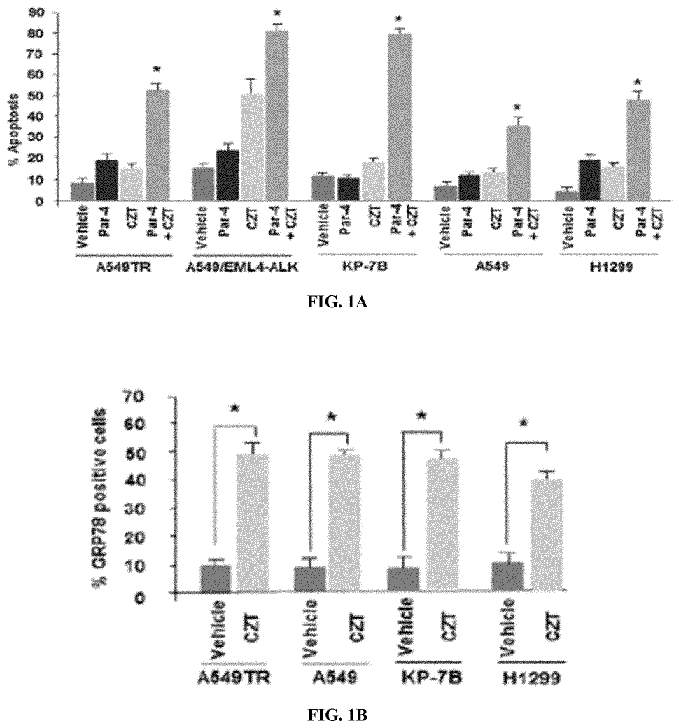

A lack of cell surface GFP78 expression may prevent secreted Par-4 from inducing apoptosis in cancer cells. In order to identify drugs that may complement the action of Par-4 by enhancing the expression of cell surface GRP78 for induction of apoptosis in lung cancer cells, an FDA-approved drug library of over 1400 compounds (from Selleck) was screened, and CZT was identified. When used together with CZT, recombinant Par-4 showed increased apoptosis in both ALK-positive (A549/EML4-ALK; from ATCC) and ALK-negative A549, A549TR, which are taxane resistant (27), p53-null H1299, and mutant-Kras, p53-null KP-7B (from Tyler Jacks, MIT, ref. 6) lung cancer cell lines (FIG. 1A). This action of CZT was associated with elevation of Par-4 receptor GRP78 on lung tumor cell surface (FIG. 1B). By contrast, CQ did not induce apoptosis in conjunction with CZT when added directly to cancer cells (FIG. 1C), implying Par-4, not autophagy-inhibition by CQ, induces apoptosis.

### Example 2: CQ and CZT Together Remarkably Inhibit Tumor Growth

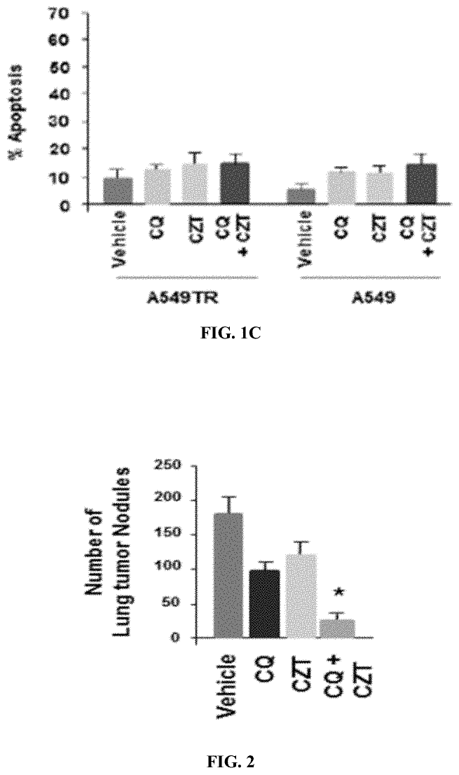

As CZT upregulates GRP78 expression on the surface of lung cancer cells), the effect of CQ, which induces Par-4 secretion in mice (ref. 6), was examined in conjunction with CZT. CQ induced significantly greater inhibition of LLC1-derived lung tumor nodules in the presence of CZT, relative to that with CZT or CQ administered separately (FIG. 2). CZT itself, unexpectedly, showed an effect in these studies.

### Example 3: CQ and CZT Together Remarkably Inhibit Tumor Growth

Since CZT upregulates GRP78 expression on the surface of lung cancer cells (FIG. 1), the effect of CQ was examined, which induces Par-4 secretion in mice, in conjunction with CZT in LLC1-derived lung tumors. CQ induced significantly greater inhibition of lung tumor nodules in the presence of CZT, relative to that with CZT or CQ administered separately (FIG. 2). As CZT itself, unexpectedly, showed an effect in these studies.

Results

CZT as a drug that upregulates Par-4 receptors on cancer cells and thereby complements the action of secreted Par-4 to sensitize ALK-negative tumors to apoptosis. Currently, CZT use is restricted to lung tumors that are ALK+/MET+/ROS1+. However, such tumors quickly become resistant to CZT. This invention expands the range of CZT action beyond tumors expressing activated ALK/MET/ROS1. Accordingly, the CQ+CZT or Par-4+CZT combination should be clinically effective against ALK+ tumors that are resistant to CZT alone, as well as in most lung tumors that lack activated ALK.

It will be understood that various details of the presently disclosed subject matter can be changed without departing from the scope of the subject matter disclosed herein. Furthermore, the foregoing description is for the purpose of illustration only, and not for the purpose of limitation.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Jessica J. Lin J J and Shaw A T. Resisting resistance: targeted therapies in lung cancer. Trends Cancer 2016; 2:350-364.
2. Devarakonda S, and Govindan R. Targeting resistance to targeted therapies: combating a resilient foe. Clin Cancer Res. 2018; 6112-6114.
3. Shrestha-Bhattarai T, and Rangnekar V M. Cancer-selective apoptotic effects of extracellular and intracellular Par-4. Oncogene 2010; 29: 3873-3880.
4. Hebbar N, Wang C, and Rangnekar V M. Mechanisms of apoptosis by the tumor suppressor Par-4. J Cell Physiol. 2012, 227: 3715-3721.
5. Burikhanov R, Zhao Y, Goswami A, Qiu S, Schwarze S R, and Rangnekar V M. The tumor suppressor Par-4 activates an extrinsic pathway for apoptosis. Cell 2009; 138: 377-388.
6. Burikhanov R, Hebbar N, Noothi S K, Shukla N, Sledziona J, Araujo N, Kudrimoti M, Wang Q J, Watt D S, Welch D R, et al. Chloroquine-inducible Par-4 secretion Is essential for tumor cell apoptosis and inhibition of metastasis. Cell Rep. 2017; 18: 508-519.
7. Wang P, Burikhanov R, Jayswal R, Weiss H L, Arnold S M, Villano J L, and Rangnekar V M. Neoadjuvant administration of hydroxychloroquine in a phase 1 clinical trial induced plasma Par-4 levels and apoptosis in tumors. Genes Cancer 2018; 9:190-197.
8. Boya P, Gonzalez-Polo R A, Poncet D, Andreau K, Vieira H L, Roumier T, Perfettini J L, and Kroemer G. Mitochondrial membrane permeabilization is a critical step of lysosome-initiated apoptosis induced by hydroxychloroquine. Oncogene 2003; 22:3927-3936.
9. Maycotte P, Aryal S, Cummings C T, Thorburn J, Morgan M J, and Thorburn A. Chloroquine sensitizes breast cancer cells to chemotherapy independent of autophagy. Autophagy 2012; 8: 200-212.
10. Maes H, Kuchnio A, Peric A, Moens S, Nys K, De Bock K, Quaegebeur A, Schoors S, Georgiadou M, Wouters J, Vinckier S, Vankelecom H, Garmyn M, Vion A C, Radtke F, Boulanger C, Gerhardt H, Dejana E, Dewerchin M, Ghesquiere B, Annaert W, Agostinis P, and Carmeliet P. Tumor vessel normalization by chloroquine independent of autophagy. Cancer Cell 2014; 26:190-206.
11. Chen D, Xie J, Fiskesund R, Dong W, Liang X, Lv J, Jin X, Liu J, Mo S, Zhang T, Cheng F, Zhou Y, Zhang H, Tang K, Ma J, Liu Y, and Huang B. Chloroquine modulates antitumor immune response by resetting tumor-associated macrophages toward M1 phenotype. Nat Commun. 2018; 9:873-887.
12. https://www.cancer.gov/abut-cancer/treatment/drugs/crizotinib
13. Devarakonda S, Ganesh B, Mann J and Govindan R. Crizotinib: an orphan drug for treating non-small-cell lung cancer. Expert Opinion on Orphan Drugs 2015; 3: 1209-1218.
14. Siegel R L, Miller K D, and Jemal A. Cancer statistics, 2018. CA Cancer J Clin. 2018; 68:7-30.
15. Goswami A, Burikhanov R, de Thonel A, Fujita N, Goswami M, Zhao Y, et al. Binding and phosphorylation of Par-4 by Akt Is essential for cancer cell survival. Molecular Cell 2005; 20:33-44.
16. Zhao Y, Burikhanov R, Brandon J, Qiu S, Shelton B J, Spear B, et al. Systemic Par-4 inhibits non-autochthonous tumor growth. Cancer Biology Therapy 2011; 12:152-157.
17. Chang S, Kim J H, and Shin J. p62 forms a ternary complex with PKCzeta and PAR-4 and antagonizes PAR-4-induced PKCzeta inhibition. FEBS Lett. 2002; 510:57-61.
18. Araujo N, Hebbar N and Rangnekar V M. GRP78 is a targetable receptor on cancer and stromal cells. EBioMedicine 2018; 33:2-3.
19. Cook J, Krishnan S, Ananth S, Sells S F, Shi Y, Walther M M, et al. Decreased expression of the pro-apoptotic protein Par-4 in renal cell carcinoma. Oncogene 1999; 18:1205-1208.
20. Moreno-Bueno G, Fernandez-Marcos P J, Collado M, Tendero M J, Rodriguez-Pinilla S M, Garcia-Cao I, et al. Inactivation of the candidate tumor suppressor Par-4 in endometrial cancer. Cancer Research 2007; 67:1927-34.
21. Alvarez J V, Pan T C, Ruth J, Feng Y, Zhou A, Pant D, Grimley J S, Wandless T J, Demichele A; I-SPY 1 TRIAL Investigators, and Chodosh L A. Par-4 downregulation promotes breast cancer recurrence by preventing multinucleation following targeted therapy. Cancer Cell 2013; 24: 30-44.
22. Shrestha-Bhattarai T, Hebbar N, and Rangnekar V M. Par(-4)oxysm in breast cancer. Cancer Cell 2013; 24: 3-5.
23. Qiu S G, Krishnan S, El-Guendy N, and Rangnekar V M. Negative regulation of Par-4 by oncogenic Ras is essential for cellular transformation. Oncogene 1999; 18: 7115-7123.
24. Pruitt K, Ulku A S, Frantz K, Rojas R J, Muniz-Medina V M, Rangnekar V M, Der C J, and Shields J M. Ras-mediated loss of the pro-apoptotic response protein Par-4 is mediated by DNA hypermethylation through Raf-independent and Raf-dependent signaling cascades in epithelial cells. J Biol Chem. 2005; 280:23363-23370.
25. Garcia-Cao I, Duran A, Collado M, Carrascosa M J, Martin-Caballero J, Flores J M, et al. Tumour-suppression activity of the proapoptotic regulator Par4. EMBO Rep 2005; 6: 577-583.
26. Joshi J, Fernandez-Marcos P J, Galvez A, Amanchy R, Linares J F, Duran A, Pathrose P, Leitges M, Cañamero M, Collado M, Salas C, Serrano M, Moscat J, and Diaz-Meco M T. Par-4 inhibits Akt and suppresses Ras-induced lung tumorigenesis. EMBO J. 2008; 27: 2181-2193.
27. Hebbar N, Burikhanov R, Shukla Ni, Qiu S, Zhao Y, Elenitoba-Johnson K S J, and Rangnekar V M. A naturally generated decoy of the prostate apoptosis response-4 protein overcomes therapy resistance in tumors. Cancer Res. 77:4039-4050, 2017.
28. Burikhanov R, Shrestha-Bhattarai T, Hebbar N, Qiu S, Zhao Y, Zambetti G, and Rangnekar V M. Paracrine apoptotic effect of p53 mediated by tumor suppressor Par-4. Cell Rep 2014; 6:271-277.
29. Rebecca V W, and Amaravadi R K. Emerging strategies to effectively target autophagy in cancer. Oncogene 2016; 35: 1-11.
30. Levy J M M, Towers C G, and Thorburn A. Targeting autophagy in cancer. Nat Rev Cancer 2017; 17: 528-542.
31. Gump, G M, and Thorburn, A. Autophagy and apoptosis—what's the connection? Trends Cell Biol. 2011; 21, 387-392.
32. Rosenfeldt M T, O'Prey J, Morton J P, Nixon C, MacKay G, Mrowinska A, Au A, Rai T S, Zheng L, Ridgway R, Adams P D, Anderson K I, Gottlieb E, Sansom O J, and Ryan K M. p53 status determines the role of autophagy in pancreatic tumour development. Nature 2013; 504: 296-300.
33. Isozaki H, Takigawa N, and Kiura K. Mechanisms of acquired resistance to ALK inhibitors and the rationale for treating ALK-positive lung cancer. Cancers (Basel). 2015; 7: 763-783.
34. Dagogo-Jack I and Shaw A T. Crizotinib resistance: implications for therapeutic strategies. Ann Oncol. 2016; 27: iii42-iii50.
35. Yoshida R, Sasaki T, Minami Y, Hibino Y, Okumura S, Sado M, Miyokawa N, Hayashi S, Kitada M, and Ohsaki Y. Activation of Src signaling mediates acquired resistance to ALK inhibition in lung cancer. Int J Oncol. 2017; 51:1533-1540.
36. Mitou G, Frentzel J, Desquesnes A, Le Gonidec S, AlSaati T, Beau I, Lamant L, Meggetto F, Espinos E, Codogno P, Brousset P, and Giuriato S. Targeting autophagy enhances the anti-tumoral action of crizotinib in ALK-positive anaplastic large cell lymphoma. Oncotarget. 2015; 6:30149-30164.
37. You L, Shou J, Deng D, Jiang L, Jing Z, Yao J, Li H, Xie J, Wang Z, Pan Q, Pan H, Huang W, and Han W. Crizotinib induces autophagy through inhibition of the STAT3 pathway in multiple lung cancer cell lines. Oncotarget. 2015; 6: 40268-40282.
38. Schroeder R D, Choi W, Hong D S, McConkey D J. Autophagy is required for crizotinib-induced apoptosis in MET-amplified gastric cancer cells. Oncotarget. 2017; 8:51675-51687.
39. Tsai Y L, Ha D P, Zhao H, Carlos A J, Wei S, Pun T K, Wu K, Zandi E, Kelly K, and Lee A S. Endoplasmic reticulum stress activates SRC, relocating chaperones to the cell surface where GRP78/CD109 blocks TGF-β signaling. Proc Natl Acad Sci USA. 2018; 115: E4245-E4254.
40. Ni M, Zhang Y and Lee A S. Beyond the endoplasmic reticulum: atypical GRP78 in cell viability, signaling and therapeutic targeting. Biochem J. 2011; 434: 181-188.
41. Lee A S. Glucose-regulated proteins in cancer: molecular mechanisms and therapeutic potential. Nat Rev Cancer 2014; 14: 263-276.

It will be understood that various details of the presently disclosed subject matter can be changed without departing from the scope of the subject matter disclosed herein. Furthermore, the foregoing description is for the purpose of illustration only, and not for the purpose of limitation.

1. A method of sensitizing a drug resistant cancer cell to chemotherapy, comprising: contacting the drug resistant cancer cell with an effective amount of an agent capable of increasing cell surface expression of GRP78.

2. The method of claim 1 wherein the agent capable of increasing cell surface expression of GRP78 is Crizotinib.

3. The method of claim 1, further comprising contacting the drug resistant cancer cell with an effective amount of an agent capable of increasing soluble prostate apoptosis response 4 (Par-4) in the drug resistant cancer cells.

4. The method of claim 3 wherein the agent capable of increasing soluble Par-4 is selected from chloroquine, hydroxychloroquine, or combinations thereof.

5. The method of claim 3, wherein the drug resistant cancer cell is a lung cancer cell.

6. The method of claim 3, wherein the drug resistant cancer cell is in a subject.

7. The method of claim 3, wherein the agent capable of increasing cell surface expression of GRP78 is Crizotinib and the agent capable of increasing soluble Par-4 is selected from chloroquine, hydroxychloroquine, or combinations thereof.

8. The method of claim 7 wherein the agent capable of increasing soluble Par-4 is chloroquine.

9. The method of claim 7, wherein the drug resistant cancer cell is in a subject.

10. The method of claim 8, wherein the drug resistant cancer cell is in a subject.

11. The method of claim 10, wherein the effective amount of Crizotinib is about 10 mg/kg, and the effective amount of chloroquine is about 25 mg/kg.
